(19) United States
(12) Patent Application Publication
(10) Pub. No.: US 2023/0397683 A1
(43) Pub. Date: Dec. 14, 2023

(54) Headwear with Practical Applications for the Ear

(71) Applicant: Richard Fryer, Dallas, TX (US)

(72) Inventor: Richard Fryer, Dallas, TX (US)

(21) Appl. No.: 17/750,272

(22) Filed: May 20, 2022

(51) Int. Cl. A42B 1/0188; A61F 11/12

(52) U.S. Cl. CPC A42B 1/0188; A61F 11/12

(57) ABSTRACT

A hat apparatus, where the hat apparatus comprises a crown, ear plugs, and cords. The cords have a crown end and an ear plug end. An ear plug is connected to the cord by the ear plug end and the cord is connected to the crown of the hat by the crown end. Further, the hat apparatus may have holders connected to the outer surface of the crown configured to hold the ear plugs or to hold the ear plugs and the cord. The cords and holders may be connected to the bill of the hat.

### FIELD OF THE DISCLOSURE

Embodiments of the present disclosure generally relate to headwear and more particularly to headwear such as hats used to hold objects such as earphones, ear protection and other related items.

### BACKGROUND

Individuals value their clothing for various reasons; however, an important characteristic is the practical aspects of an article of clothing. The practical aspects of an article of clothing are even more important when the clothing is used to improve the safety of the individual wearing the clothing. A number of jobs and recreational activities that involve people listening to devices using earphones or protecting their ability to hear by using ear protection that is inserted into the ear is large and growing.

### SUMMARY OF THE CLAIMED EMBODIMENTS

In one aspect, embodiments disclosed herein relate to a hat apparatus, where the hat apparatus comprises a crown, ear plugs, and cords. The cords have a crown end and an ear plug end. An ear plug is connected to the cord by the ear plug end and the cord is connected to the crown of the hat by the crown end.

In another aspect, embodiments disclosed herein relate to a hat apparatus, where the hat apparatus comprises a crown, ear plugs, and cords. The cords have a crown end and an ear plug end. An ear plug is connected to the cord by the ear plug end and the cord is connected to the crown of the hat by the crown end. The hat apparatus has holders connected to the outer surface of the crown and configured to hold the ear plugs or to hold the ear plugs and the cords.

In another aspect, embodiments disclosed herein relate to a hat apparatus, where the hat apparatus comprises a crown, a bill, ear plugs, and cords. The cords have a bill end and an ear plug end. An ear plug is connected to the cord by the ear plug end and the cord is connected to the bill of the hat by the bill end. The hat apparatus has holders connected to the bill and configured to hold the ear plugs or to hold the ear plugs and the cords.

Other aspects and advantages will be apparent from the following description and the appended claims.

### BRIEF DESCRIPTION OF DRAWINGS

FIG. 1A is a diagram that illustrates a hat according to one or more embodiments.

FIG. 1B is a diagram that illustrates a partial view of a hat according to one or more embodiments.

FIG. 2 is a diagram that illustrates an ear plug according to one or more embodiments.

FIG. 3 is a diagram that illustrates a hat according to one or more embodiments.

FIG. 4 is a diagram that illustrates a hat according to one or more embodiments.

FIG. 5A is a diagram that illustrates a hat according to one or more embodiments.

FIG. 5B is a diagram that illustrates a hat according to one or more embodiments.

FIG. 5C is a diagram that illustrates a hat according to one or more embodiments.

FIG. 6 is a diagram that illustrates a hat according to one or more embodiments.

### DETAILED DESCRIPTION

The current invention gives a user the ability to wear different varieties of headwear (often times hats and caps) and have audio earphones or ear protection attached to the headwear. The audio earphones or ear protection may be used to protect the ears from loud sound and noises. The audio earphones (audio ear plugs) may also be configured to communicate noise from the area around the user as well as potentially play music from another device that is connected wirelessly. The attached headwear improves the experience of the user because the attached audio ear plugs or ear protection (collectively referred to as ear plugs) will not be misplaced or lost thereby allowing the user to enjoy and carry out their professional responsibilities or recreational activity. Further, it is inconvenient to retrieve ear plugs from shirt or pant pockets because shirt or pant pockets may contain other objects. In certain activities the pockets may contain sharp objects that have the potential of causing injuries to the hand of the user.

The ear plugs may be attached to the headwear or headgear in a variety of ways using cords. Several types of materials may be used to make the headwear or headgear and the ear plugs. Further, the headwear or headgear may contain holders that are useful in storing or securing the ear plugs and also in allowing easy access to the ear plugs.

The headgear or headwear may be referred to as hat apparatus or a hat; however, the term encompasses all types of headgear including, but not limited to, baseball caps, performance hats, bucket hats, visors.

Various illustrative embodiments of the disclosed subject matter are described. In the interest of clarity, not all features of an actual implementation are described in this specification. It will of course be appreciated that in the development of any such actual embodiment, numerous implementation-specific decisions must be made to achieve the specific goals of the developers, such as compliance with system-related and business-related constraints, which will vary from one implementation to another. Moreover, it will be appreciated that such a development effort might be complex and time-consuming but may be a routine undertaking for those of ordinary skill in the art having the benefit of this disclosure.

The present subject matter will now be described with reference to the attached figures. Various structures, systems, and devices are schematically depicted in the drawings for purposes of explanation only and so as to not obscure the present disclosure with details that are well known to those skilled in the art. Nevertheless, the attached drawings are included to describe and explain illustrative examples of the present disclosure. The words and phrases used should be understood and interpreted to have a meaning consistent with the understanding of those words and phrases by those skilled in the relevant art. No special definition of a term or phrase, that is, a definition that is different from the ordinary and customary meaning as understood by those skilled in the art, is intended to be implied by consistent usage of the term or phrase. To the extent that a term or phrase is intended to have a special meaning, that is, a meaning other than that understood by skilled artisans, such a special definition will be expressly set forth in the specification in a definitional manner that directly and unequivocally provides the special definition for the term or phrase. With reference to the attached figures, various illustrative embodiments of the systems, devices and methods disclosed will now be described in more detail.

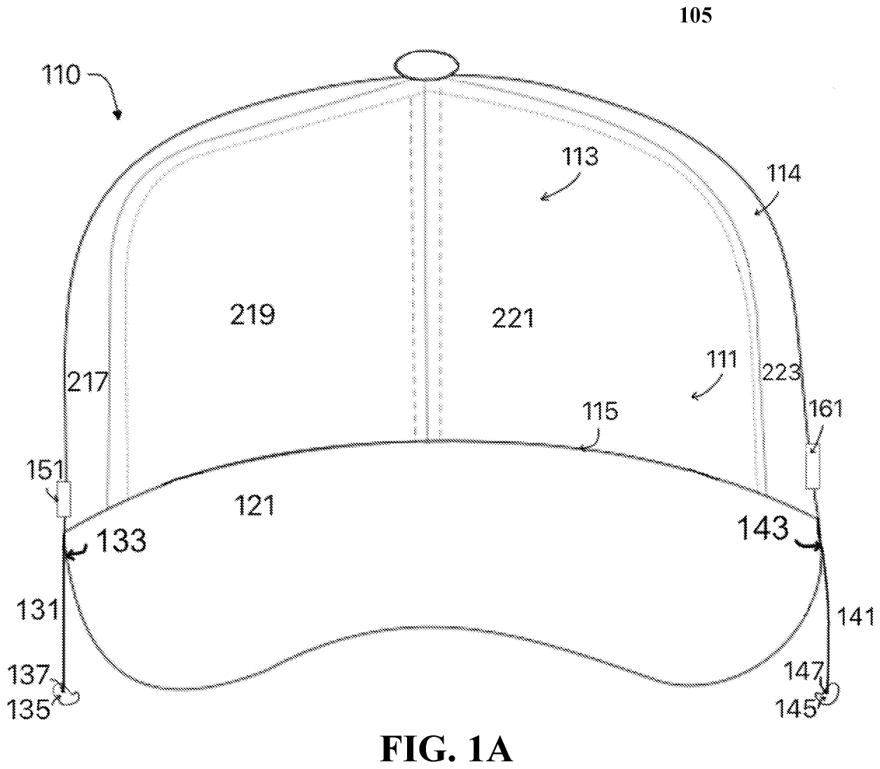

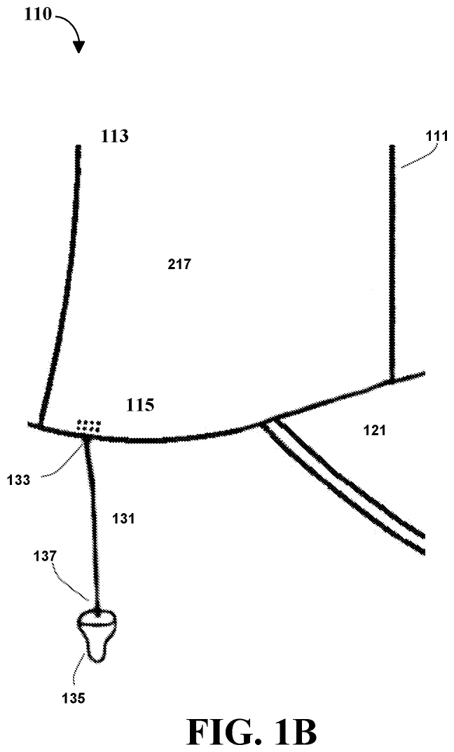

FIG. 1A is a diagram that illustrates a view of a hat appuratus 110 according to one or more embodiments. Referring to FIG. 1A, the hat may include a crown 111. The crown may be configured to have a top portion 113 and a bottom portion 115. The bottom portion may be near the opening in the crown 111. The crown 111 may be made of one or more crown segments 217, 219, 221, 223 that may be configured to form a dome-like structure that may fit over a user's head (all the segments are not shown). The crown may have an outer surface 114 and an inner surface (not shown). The inner surface of the crown is configured to be in contact with the head of a user, and the outer surface 114 is configured to be exposed to the outside environment 105. According to one or more embodiments, the crown segments 217, 219, 221, 223 that form the crown 111 may be constructed from several different materials or fabrics, including but not limited to, cotton, polyester, and spandex. Those having skill in the art will appreciate that several different types of fabrics or materials may be used to make the crown. Those having skill in the art will also appreciate that the group of materials listed may be selected and combined with other materials that may or may not be listed.

According to one or more embodiments, the hat may include a bill 121 where the bill may be attached to the bottom portion 115 of the crown 111. The bill may be fabricated from a similar materials or fabrics as the crown; however according to one or more embodiments the crown 111 may be made from other materials or fabrics that would be appreciated by those having skill in the art. According to one or more embodiments, the bill 121 may protrude away from the crown 111 at an incline. In one or more embodiments, the bill 121 may be a curved plane or a flat plane.

According to one or more embodiments, the hat 110 may be configured to include a first cord 131 that is connected to the crown 111 by a first crown end 133 of the first cord 131, and the first cord 131 is connected to a first ear plug 135 by the first ear plug end 137 of the first cord 131. According to one or more embodiments, the hat 110 is configured to include a second cord 141 that is connected to the crown 111 by a second crown end 143 of the second cord 141, and the second cord 141 is connected to a second ear plug 145 by the second ear plug end 147 of the second cord 141. The ear plugs 135, 145 are configured to fit into the ears of a user. According to one or more embodiments, the hat 110 may be configured to include a first holder 151 and a second holder 161 that is connected to the crown 111 of the hat. The holders 151, 161 may be configured to hold or store the cords and/or the earplugs. For example, the first holder 151 may be configured to hold the first cord 131 and/or the first ear plug 135, and the second holder 161 may be configured to hold the second cord 141 and/or the second ear plug 145. In one or more embodiments a single holder may be configured to hold both the first cord 131 or the first ear plug 135 and the second cord 141 or the second ear plug 145. A holder configured to hold both a cord and an ear plug may be configured to hold a part of the cord and/or a part of the earplug. According to one or more embodiments, the holders 151, 161 may be connected to the outer surface 114 of the crown 111.

FIG. 1B is a diagram that illustrates a partial view of a hat apparatus 110 according to one or more embodiments. Referring to FIG. 1B, the hat 110 may have a crown 111 that has may be configured to have a top portion 113 and a bottom portion 115. The hat 110 may be configured to include a cord 131 that is connected to the crown 111 by a crown end 133 of the cord 131, and the cord 131 is connected to an ear plug 135 by the ear plug end 137 of the cord 131. The location of the connection of the crown end 133 of the cord 131 may vary on the crown. According to one or more embodiments, the location of the connection of the crown end 133 of the cord 131 may be near the bottom portion 115 of a crown 111 on a crown segment 217 near the ear of a user configured to allow the user accessibility to the ear plug.

Referring to FIG. 1A, the cords 131, 141 may be configured to have various lengths and widths. The length of the cords may vary depending on factors including the head size of the user. According to one or more embodiments, the cords 131, 141 may be around 1.0 inch in length or greater.

The cords 131, 141 are made of various materials that are used to connect the ear plugs 135, 145 to the crown 111. In one or more embodiments, the cords 131, 141 may be made of materials, including but not limited to, metal, silicone, rubber, rope, plastic, foam, fiber, or cloth. Those having skill in the art will appreciate that several different types of materials may be used to make the cords that connect the crown of the hat to the ear plugs. Those having skill in the art will also appreciate that the group of materials listed may be selected and combined with other materials that may or may not be listed.

The cords 131, 141 may be connected on various locations on crown. According to one or more embodiments, the crown ends 133, 143 of the cords 131, 141 may be permanently connected to the crown 111 by being sewn to a location on the crown. In one or more embodiments, the cords may be connected on the outer surface 114 of the crown 111. In one or more embodiments, the crown ends of the cords may be connected on the inner surface of the crown 111. The crown ends of the cords may be connected between the crown and an additional fabric, such as a sweatband. According to one or more embodiments, the crown ends 133, 143 of the cords 131, 141 may be permanently connected by an adhesive to a location on the crown 111. Those having skill in the art will appreciate that several different types of materials and methods may be used to connect the cords to the crown of the hat.

According to one or more embodiments, the crown ends 133, 143 of the cords 131, 141 may be configured be removable or detachable from the crown 111 of the hat 110. The detachable cords may be achieved by having the crown ends 133, 143 of the cords 131, 141 connected to the crown 111 by materials, including but not limited to, adhesive, velcro, material with magnetic properties, or other similar means that would be appreciated by those having skill in the art. Those having skill in the art will also appreciate that the group of materials listed may be selected and combined with other materials that may or may not be listed. According to one or more embodiments, the cords may be detached from the crown and stored in a holder.

According to one or more embodiments, the ear plugs 135, 145 may be permanently connected to the cords 131, 141 by the ear plug ends 137, 147 by methods that would be appreciated by those having skill in the art will appreciate. However, according to one or more embodiments the ear plugs 135, 145 may be configured to be detachable from the cords 131, 141 by the ear plug ends 137, 147. Detachable ear plugs maybe useful in replacing damaged or defective ear plugs. Those having skill in the art will appreciate that several different items may be used to make the ear plugs 135, 145 detachable from the cords 131, 141 by the ear plug ends, including but not limited to, sewing, glue, buttons, hooks or magnets or other similar means that would be appreciated by those having skill in the art.

According to one or more embodiments the ear plugs 135, 145 may be types of ear protection that are configured to fit into the ears of a user. According to one or more embodiments, the ear plugs 135, 145 may be types of audio ear plugs that are configured to fit into the ears of a user. The audio ear plugs may be configured to communicate audio from a device to an audio ear plug. The audio ear plugs may communicate the audio without a physical connection from the cord such as types of wireless connectivity including Bluetooth connectivity. Both the ear protection and the audio ear plugs may be made of materials, including but not limited to, silicone, rubber, rope, plastic, foam, metal, or cloth. Those having skill in the art will appreciate that several different types of materials may be used to make the ear protection that fits into the ears of a user. Those having skill in the art will also appreciate that the group of materials listed may be selected and combined with other materials that may or may not be listed.

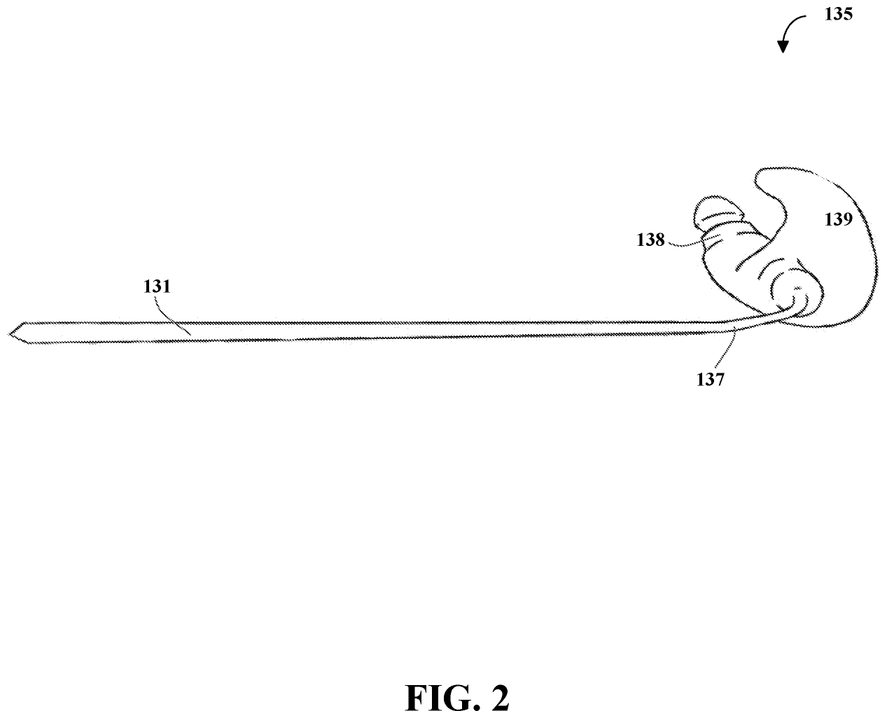

FIG. 2 is a diagram that illustrates a view of an ear plug 135 according to one or more embodiments. Referring to FIG. 2, an ear plug 135 may be connected to a cord 131 by the first ear plug end 137 of the cord 131. An ear plug 135 may have various shapes or designs. According to one or more embodiments, a first part 138 of the ear plug 135 may be occupy in a portion of the ear canal of the user, and a second part 139 of the ear plug 135 may occupy a portion of the concha (the area of the ear outside the ear canal) of the user.

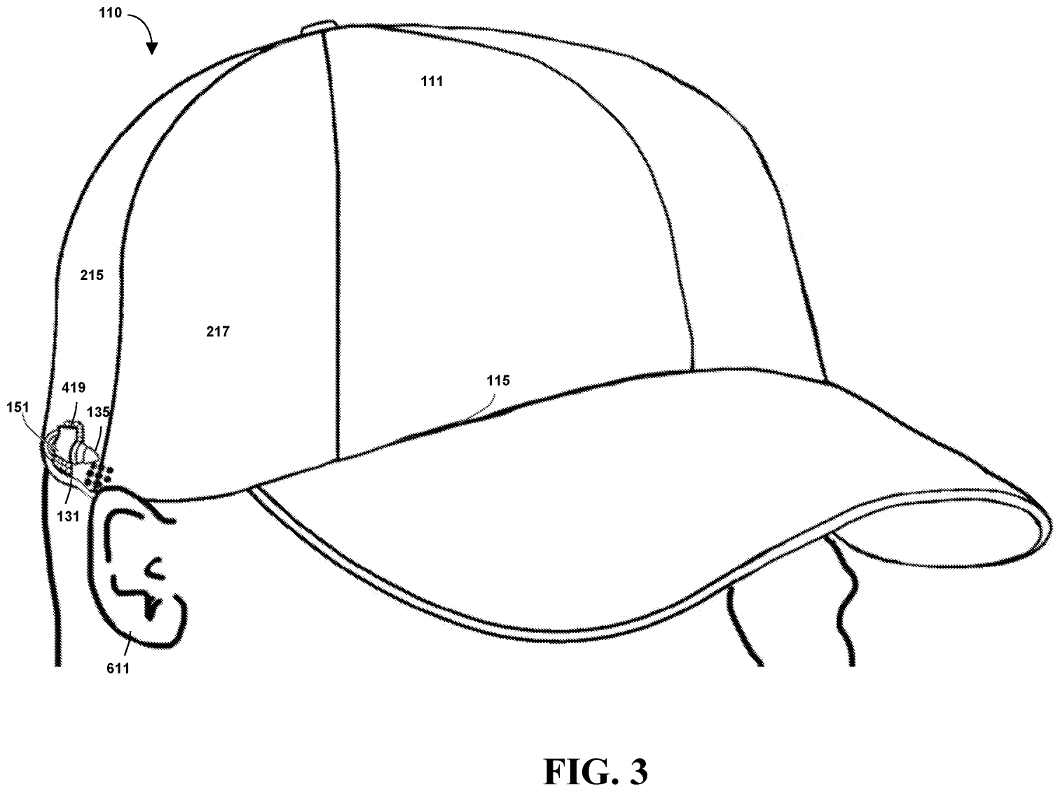

FIG. 3 is a diagram that illustrates a view of a hat apparatus 110 according to one or more embodiments. Referring to FIG. 3, a holder 151 may be connected to the hat 110. The location of the holder 151 may vary on the crown 111. According to one or more embodiments, the holder may be connected near the bottom portion 115 of a crown 111 on a crown segment 215 near the ear 611 of a user. The holder 151 may be configured to hold or store a cord 131 and an ear plug 135. As shown in FIG. 3, the holder 151 may be configured to hold the cord 131 in a folded or wrapped position. In one or more embodiments, the holder may include a strap that is configured to secure the cord 131 and the ear plug 135. As shown in FIG. 3, a holder 151 that includes a strap may use Velcro 419 parts connected a crown segment 215 to secure the cord 131 and the ear plug 135. Those having skill in the art will appreciate that several different types of components of a holder may be configured to hold or secure the cord and the ear plug, including but not limited to, Velcro, buttons, hooks and magnets. Those having skill in the art will also appreciate that the group of items listed may be selected and combined with other items that may or may not be listed.

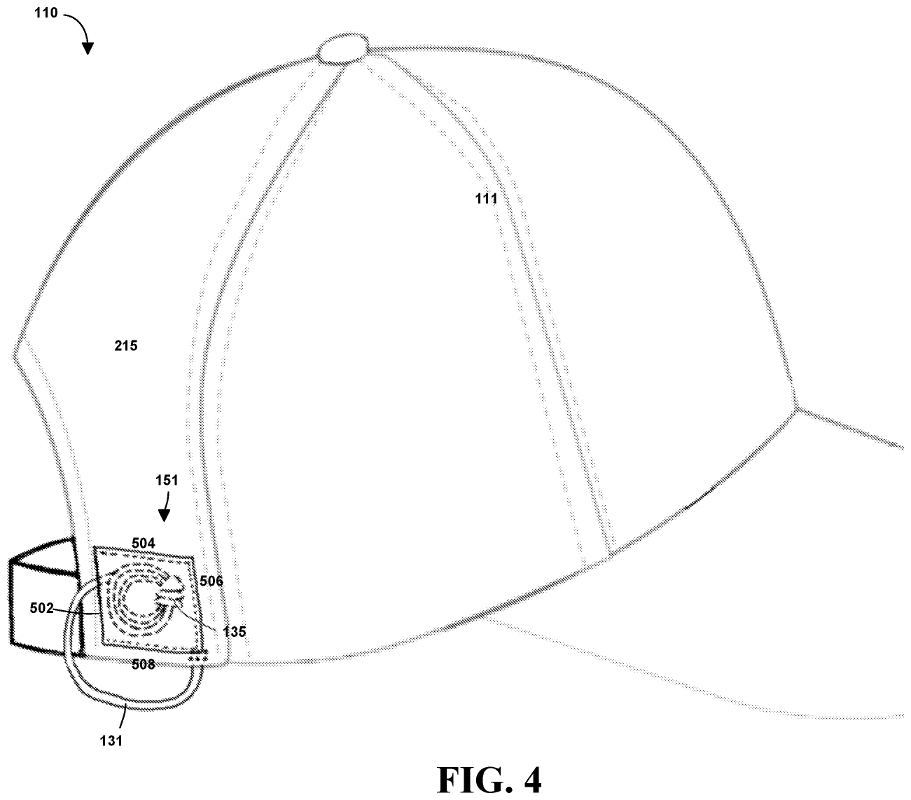

FIG. 4 is a diagram that illustrates a view of a hat apparatus 110 according to one or more embodiments. Referring to FIG. 4, a holder 151 may be connected to the hat 110. The holder 151 may be configured to hold or store an ear plug 135 and a cord 131. As shown in FIG. 4, the holder 151 may be configured to hold the ear plug 135 and the cord 131 in a folded position. In one or more embodiments, the holder may be a storage component that is configured to contain the cord 131 and the ear plug 135. The holders that are storage components are configured to store and keep the earplugs and the cords in a safe and secure location out of harm's way. A holder 151 that is a storage component may be connected to a crown segment 215 to secure the cord 131 and the ear plug 135. According to one or more embodiments, the storage may be configured to have a rectangular shape with a first side 502, a second side 504, a third side 506, and a four side 508. One or more of the sides may have an opening or cavity that allows the insertion of the ear plug 135 and the cord 131. For example, the first side 502 may have an opening that allows the insertion of the ear plug 135 and the cord 131, and the second side 504, the third side 506, and a four side 508 may not have an opening. According to one or more embodiments, a holder 151 may be detachable from the hat by means that would be appreciated by those having skill in the art. Those having skill in the art will further appreciate that several different types of materials may be used to make a holder 151 that is a storage components, including but not limited to metal, rope fabric, polyester, or cotton. Those having skill in the art will also appreciate that the group of items listed may be selected and combined with other items that may or may not be listed.

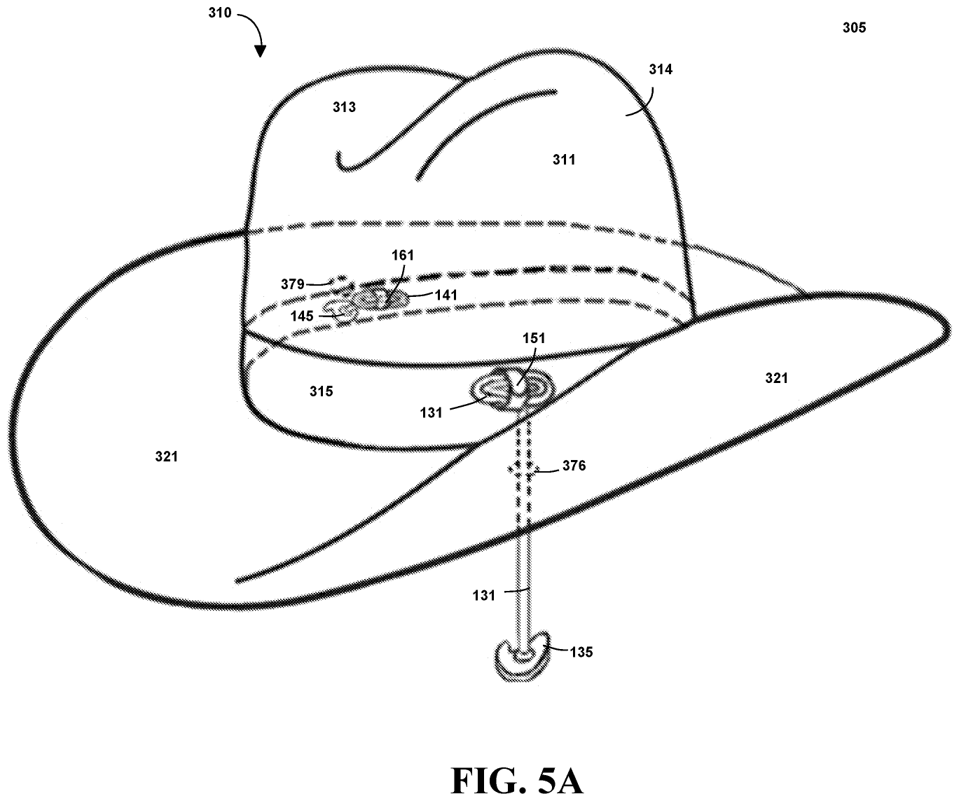

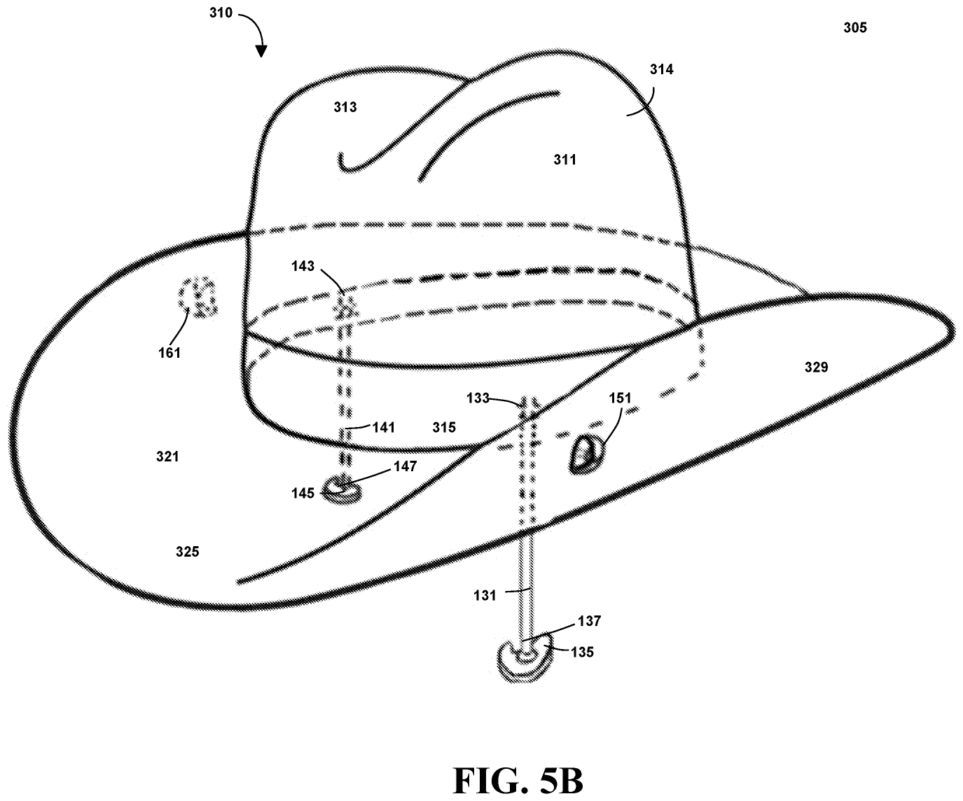

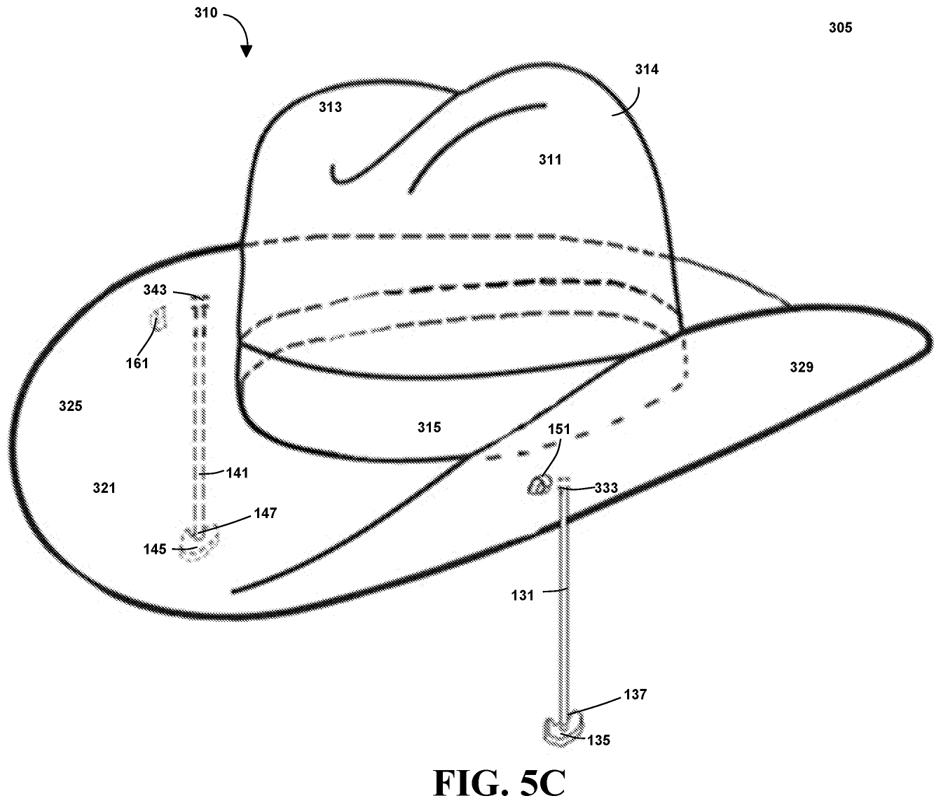

FIG. 5A is a diagram that illustrates a view of a hat apparatus 310 according to one or more embodiments. Referring to FIG. 5A, the hat may include a crown 311. The crown may be configured to have a top portion 313 and a bottom portion 315. The bottom portion 315 may be near the opening in the crown 311. The crown 311 may be configured to form a dome-like structure. The crown may have an outer surface 314 and an inner surface (not shown). The inner surface of the crown is configured to be in contact with a user's head and the outer surface 314 is configured to be exposed to the environment 305.

According to one or more embodiments, the hat 310 may include a bill 321 where the bill may be connected to the bottom portion 315 of the crown 311. According to one or more embodiments, the bill 321 may protrude away from the crown 311. In one or more embodiments, the bill 321 may be configured as a wide flat brim or wide curved brim that is connected along the circumference of the bottom portion 315 of the crown 311.

According to one or more embodiments, the bill 321 may have one or more holes near the crown 311 that is configured to allow a cord 131 or ear plug 135 to pass through the bill 321. The cord 131 or the ear plug 135 may be passed through a first hole 376 in the bill 321 to allow the cord 131 or the ear plug 135 useful access to the holder 151 and to the ear of a user. A second cord 141 or the ear plug 145 may be passed through a second hole 379 in the bill 321 to allow the second cord 141 or the ear plug 145 useful access to a second holder 161 and to the ear of a user.

FIG. 5B is a diagram that illustrates a view of a hat apparatus 310 according to one or more embodiments. Referring to FIG. 5B, the hat may include a crown 311. The crown may be configured to have a top portion 313 and a bottom portion 315. The bottom portion may be near the opening in the crown 311. The crown 311 may be configured to form a dome-like structure. The crown may have an outer surface 314 and an inner surface (not shown). The inner surface of the crown is configured to be in contact with a user's head and the outer surface 314 is configured to be exposed to the environment 305.

According to one or more embodiments, the hat 310 may include a bill 321 where the bill may be connected to the bottom portion 315 of the crown 311. The bill may have a top bill side 325 and a bottom bill side 329. The top bill side 325 is generally configured to face the crown 311 and the bottom bill side 329 is generally configured to face the user of the hat 310. According to one or more embodiments, the bill 321 may protrude away from the crown 311. In one or more embodiments, the bill 321 may be configured as a wide flat brim or wide curved brim that is connected along the circumference of the bottom portion 315 of the crown 311.

According to one or more embodiments, the hat 310 may be configured to include a first cord 131 that is connected to the crown 311 by a first crown end 133 of the first cord 131, and the first cord 131 is connected to a first ear plug 135 by the first ear plug end 137 of the first cord 131. According to one or more embodiments, the hat 310 is configured to include a second cord 141 that is connected to the crown 111 by a second crown end 143 of the second cord 141, and the second cord 141 is connected to a second ear plug 145 by the second ear plug end 147 of the second cord 141. According to one or more embodiments, the hat 110 may be configured to include a first holder 151 and a second holder 161 that is connected to the bill 321 of the hat. According to one or more embodiments, the holders 151, 161 may be connected to the bottom bill side 329 of the bill 321 as shown in FIG. 5B. However, according to one or more embodiments, the holders may be connected to the top bill side 325 of the bill 321.

FIG. 5C is a diagram that illustrates a view of a hat apparatus 310 according to one or more embodiments. Referring to FIG. 5C, the hat may include a crown 311. The crown may be configured to have a top portion 313 and a bottom portion 315. The bottom portion may be near the opening in the crown 311. The crown 311 may be configured to form a dome-like structure. The crown may have an outer surface 314 and an inner surface (not shown). The inner surface of the crown is configured to be in contact with a user's head and the outer surface 314 is configured to be exposed to the environment 305.

According to one or more embodiments, the hat 310 may include a bill 321 where the bill may be connected to the bottom portion 315 of the crown 311. The bill may have a top bill side 325 and a bottom bill side 329. The top bill side 325 is generally configured to face towards the crown 311 and the bottom bill side 329 is generally configured to face the towards user of the hat 310. According to one or more embodiments, the bill 321 may protrude away from the crown 311. In one or more embodiments, the bill 321 may be configured as a wide flat brim or wide curved brim that is connected along the circumference of the bottom portion 315 of the crown 311.

According to one or more embodiments, the hat 310 may be configured to include a first cord 131 that is connected to the bill 321 by a first bill end 333 of the first cord 131, and the first cord 131 is connected to a first ear plug 135 by the first ear plug end 137 of the first cord 131. According to one or more embodiments, the hat 310 is configured to include a second cord 141 that is connected to the bill 321 by a second bill end 343 of the second cord 141, and the second cord 141 is connected to a second ear plug 145 by the second ear plug end 147 of the second cord 141. According to one or more embodiments, the bill ends 333, 343 of the cords 131, 141 may be connected to the bottom bill side 329 of the bill 321 as shown in FIG. 5C. However, according to one or more embodiments, the bill ends 333, 343 of the cords 131, 141 may be connected to the top bill side 325 of the bill 321.

According to one or more embodiments, the hat 310 may be configured to include a first holder 151 and a second holder 161 that is connected to the bill 321 of the hat. According to one or more embodiments, the holders 151, 161 may be connected to the bottom bill side 329 of the bill 321 as shown in FIG. 5B. However, according to one or more embodiments, the holders may be connected to the top bill side 325 of the bill 321.

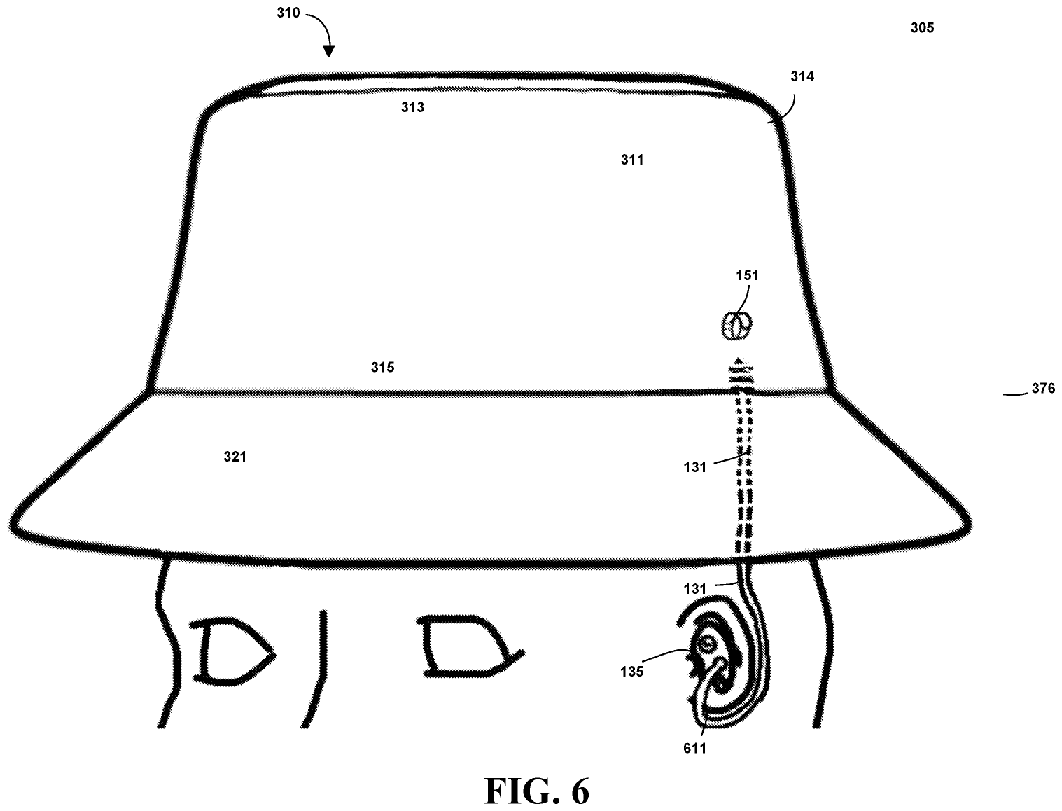

FIG. 6 is a diagram that illustrates a view of a hat apparatus 310 according to one or more embodiments. Referring to FIG. 6, the hat may include a crown 311. The crown may be configured to have a top portion 313 and a bottom portion 315. The bottom portion may be near the opening in the crown 311. The crown 311 may be configured to form a dome-like structure. The crown may have an outer surface 314 and an inner surface (not shown). The inner surface of the crown is configured to be in contact with a user's head and the outer surface 314 is configured to be exposed to the environment 305.

According to one or more embodiments, the hat 310 may include a bill 321 where the bill may be connected to the bottom portion 315 of the crown 311. The bill 321 may be connected along the circumference of the bottom portion 315 of the crown 311. According to one or more embodiments, the bill 321 may protrude away from the crown 311. In one or more embodiments, the bill 321 may be configured in as wide flat brim that slants towards the ear 611 of the user.

According to one or more embodiments, the cord 131 may be attached near the bottom portion 315 of the inner surface of the crown 311 above the ear 611 of the user. A holder 151 configured to hold the ear plug 135, cord 131 or ear plug 135 may be attached to the outer surface 314 of the crown 311. After use, the ear plug 135, or the cord 131 may be removed from the ear 611 of the user and stored in the holder 151.

Unless defined otherwise, all technical and scientific terms used have the same meaning as commonly understood by one of ordinary skill in the art to which these systems, apparatuses, methods, processes and compositions belong.

The singular forms “a,” “an,” and “the” include plural referents, unless the context clearly dictates otherwise.

As used here and in the appended claims, the words “comprise,” “has,” and “include” and all grammatical variations thereof are each intended to have an open, non-limiting meaning that does not exclude additional elements or steps.

“Optionally” means that the subsequently described event or circumstances may or may not occur. The description includes instances where the event or circumstance occurs and instances where it does not occur.

When the word “approximately” or “about” are used, this term may mean that there can be a variance in value of up to ±30%.

Ranges may be expressed as from about one particular value to about another particular value, inclusive. When such a range is expressed, it is to be understood that another embodiment is from the one particular value to the other particular value, along with all particular values and combinations thereof within the range.

While the disclosure includes a limited number of embodiments, those skilled in the art, having benefit of this disclosure, will appreciate that other embodiments may be devised which do not depart from the scope of the present disclosure. Accordingly, the scope should be limited only by the attached claims.

1. A hat apparatus, where the hat apparatus comprises:
a crown;
a first ear plug configured to fit in the ear of a user;
a second ear plug configured to fit in the ear of a user;
a first cord having a first crown end and a first ear plug end;
a second cord having a second crown end and a second ear plug end;
wherein the first ear plug is connected to the first cord by the first ear plug end and the first cord connected to the crown by the first crown end;
wherein the second ear plug is connected to the second cord by the second ear plug end and the second cord connected to the crown by the second crown end.

2. The hat apparatus of claim 1, wherein the cords comprise of material including, but not limited to, metal, silicone, rubber, rope, plastic, foam, fiber or cloth.

3. The hat apparatus of claim 1, wherein the cords are detachable from the crown ends.

4. The hat apparatus of claim 1, wherein the cords are detachable from the ear plug ends.

5. The hat apparatus of claim 1, wherein the cords have a length of about 1.0 inch or longer.

6. The hat apparatus of claim 1, wherein the ear plugs have a first part of the ear plug configured to occupy in a portion of an ear canal of the user a second part of the ear plug configured to occupy a concha of the user.

7. The hat apparatus of claim 1, wherein the ear plugs are configured to be ear protection.

8. The hat apparatus of claim 1, wherein the ear plugs are configured to be audio earphones.

9. The hat apparatus of claim 1, wherein the ear plugs comprise of material including, but not limited to, silicone, rubber, rope, plastic, foam, metal, or cloth.

10. The hat apparatus of claim 1, wherein a holder is connected to an outer surface of the crown to hold the first ear plug and the second ear plug.

11. The hat apparatus of claim 1, wherein the outer surface of the crown further comprises a first holder configured to hold the first ear plug and a second holder configured to hold the second ear plug.

12. The hat of claim 1, wherein the outer surface of the crown further comprises a first holder configured to hold the first ear plug and the first cord, and a second holder configured to hold the second ear plug and the second cord.

13. The hat apparatus of claim 1, wherein the hat further comprises a bill connected to a bottom portion of the crown.

14. The hat apparatus of claim 13, wherein the bill has first hole and a second hole configured to allow the cord or ear plug to be passed through.

15. A hat apparatus, where the hat apparatus comprises:
a crown having an inner surface and an outer surface and a bottom portion;
a bill connected to a bottom portion of the crown;
a first ear plug configured to fit in the ear of a user;
a second ear plug configured to fit in the ear of a user;
a first cord having a first crown end and a first ear plug end;
a second cord having a second crown end and a second ear plug end;
a first holder configured to hold the first ear plug and the first cord;
a second holder configured to hold the second ear plug and the second cord;
wherein the first ear plug is connected to the first cord by the first ear plug end and the first cord connected to the crown by the first crown end;
wherein the second ear plug is connected to the second cord by the second ear plug end and the second cord connected to the crown by the second crown end;
Wherein the holders are connected to the outer surface of the crown.

16. The hat of claim 15, wherein the holders are storage components configured to store and keep the earplugs and the cords in a safe and secure location.

17. The hat of claim 15, wherein the holders comprise Velcro configured to store and keep the earplugs and the cords in a safe and secure location.

18. The hat of claim 15, wherein the holders comprise of material including, but not limited to, metal, rope fabric, polyester, or cotton.

19. A hat apparatus, where the hat apparatus comprises:
a crown;
a bill connected to a bottom portion of the crown;
a first ear plug configured to fit in the ear of a user;
a second ear plug configured to fit in the ear of a user;
a first cord having a first bill end and a first ear plug end;
a second cord having a second crown end and a second ear plug end;
wherein the first ear plug is connected to the first cord by the first ear plug end and the first cord connected to the bill by the first bill end;
wherein the second ear plug is connected to the second cord by the second ear plug end and the second cord connected to the crown by the second crown end.

20. The hat of claim 19, wherein the bill further comprises a first holder configured to hold the first ear plug and a part of the first cord, and a second holder configured to hold the second hear plug and a part of the second cord.
